(19) United States
(12) Patent Application Publication
(10) Pub. No.: US 2012/0269827 A1
(43) Pub. Date: Oct. 25, 2012

(54) Compositions and Methods for Treatment of Ovarian, Peritoneal, and Fallopian Tube Cancer

(75) Inventors: Kathleen R. WHITEMAN, Wilmington, MA (US); James J. O'Leary, Newton, MA (US); Robert John Lutz, Wayland, MA (US)

(73) Assignee: ImmunoGen, Inc., Waltham, MA (US)

(21) Appl. No.: 13/434,419

(22) Filed: Mar. 29, 2012

Related U.S. Application Data
(60) Provisional application No. 61/470,676, filed on Apr. 1, 2011.

(51) Int. Cl. A61K 39/44; A61P 35/00

(52) U.S. Cl. 424/178.1

(57) ABSTRACT

The present invention relates to highly effective anti-cancer drug combinations, pharmaceutical compositions comprising the same, and uses thereof in the treatment of ovarian, peritoneal, or fallopian tube cancer. In particular, the present invention is based on the discovery that the administration of a CD56 antibody linked to a cytotoxic compound (e.g., an immunoconjugate) in combination with a chemotherapeutic agent (in particular a gemcitabine compound, a topotecan compound, and a doxorubicin compound), improves the therapeutic index in the treatment of ovarian, peritoneal, or fallopian tube cancer over and above the additive effects of the anticancer agents used alone. In one embodiment of the invention, combinations of the CD56 antibody, or fragment thereof, linked to a cytotoxic compound plus an additional chemotherapeutic agent have a synergistic effect in the ovarian cancer therapeutic index.

### CROSS REFERENCE TO RELATED APPLICATIONS

This application claims the priority benefit of U.S. Provisional Application No. 61/470,676, filed Apr. 1, 2011, which is incorporated by reference herein in its entirety.

### REFERENCE TO SEQUENCE LISTING SUBMITTED ELECTRONICALLY VIA EFS-WEB

The content of the electronically submitted Sequence Listing (Name: SequenceListing.txt; Size 6,310 bytes; and Date of Creation Mar. 29, 2012) is herein incorporated by reference in its entirety.

### BACKGROUND OF THE INVENTION

Ovarian cancer is the most common cancer of the female reproductive tract, presenting an estimated 22,430 new cases and 15,280 deaths in the United States in 2007 (Jemal et al., C A Cancer. J. Clin. 2007, 57(1):43-56). Approximately 70% of ovarian cancers are diagnosed at advanced stage and only 30% of women with such cancers can expect to survive 5 years (Cho and Shih, Annu. Rev. Pathol. 2009, 4:284-313).

Current treatments for ovarian cancer include surgery, radiation therapy, chemotherapy, and combinations thereof. The standard first-line chemotherapy for ovarian cancer is a combination of a taxane and a platinum-containing drug. However, such combinations present toxicity risks for patients, and resistance to cytotoxic chemotherapy is the main cause of therapeutic failure and death in women suffering from ovarian carcinoma. See, e.g., Lage and Denkert, Recent Results Cancer Res. 2007, 176:51-60. Furthermore, advanced ovarian cancer treatment with a platinum agent in combination with a taxane is currently limited by a 5-year survival rate of approximately 45%. See, e.g., March et al, Journal of Clinical Oncology, 2007, 25(29):4528-4535.

Xenograft models, e.g., where ovarian cancer cells have been injected either subcutaneously or into the peritoneal cavity, have been used extensively for the testing of novel therapeutics or modified regimens for administration of standard chemotherapeutic drugs. See, e.g., Vanderhyden et al., Reproductive Biology and Endocrinology, 2003, 1:67.

Anti-cancer drugs with different mechanisms of killing, e.g., having different targets in the cell, have been used in combination. For example, combinations of a maytansinoid immunoconjugate comprising a maytansinoid compound (e.g., DM1) linked to a monoclonal antibody (e.g., an anti-CD56 antibody) and (1) paclitaxel, (2) cisplatin and etoposide, (3) docetaxel were used in the small cell lung cancer (SCLC) xenograph model as disclosed in U.S. Pat. Nos. 7,303,749 and 7,601,354, which are incorporated herein by reference in their entirety. In addition, combinations of a maytansinoid immunoconjugate comprising a maytansinoid compound linked to a monoclonal antibody and (1) a proteasome inhibitor (bortezomib), (2) an immunomodulatory agent/anti-angiogenic agent (thalidomide or lenalidomide), or (3) a DNA alkylating agent (melphalan), with the optional further addition of a corticosteroid (dexamethasone) were used in the multiple myeloma xenograph model.

In experimental systems where anti-cancer drugs with different mechanisms of killing are combined, it has been observed that the anti-cancer drugs with independent targets (mutually exclusive drugs) either behave in an additive, synergistic, or antagonistic manner. Chou and Talalay developed a mathematical method to accurately describe such experimental results in a qualitative and quantitative manner (Chou and Talalay, Adv. Enzyme Regul. 1984, 22:27-55). Chou and Talalay showed that a combination of two mutually exclusive drugs will show the same type of effect over the whole concentration range, that is, the combination will show an additive, a synergistic, or an antagonistic type of effect. Most drug combinations show an additive effect. In some instances, however, the combination shows less or more than an additive effect. These combinations are called antagonistic or synergistic, respectively. Antagonistic or synergistic effects are generally considered unpredictable, and are unexpected experimental findings. See Knight et al., BMC Cancer 2004, 4:83; T. H. Corbett et al., Cancer Treatment Reports, 1982, 66:1187; and Tallarida, J. Pharmacol. Exp. Ther., 2001 298(3):865-72.

There is a need in the art for new and more effective methods for treating ovarian cancer. Furthermore, there is still a need for finding drug combinations that show synergism and can be effectively used for the treatment and prevention of cancer, e.g., ovarian cancer. The present invention is directed to such methods and drug combinations.

### BRIEF SUMMARY OF THE INVENTION

The present invention relates to anti-cancer combinations, pharmaceutical compositions comprising the same, and the use thereof in the treatment of ovarian, peritoneal, or fallopian tube cancer. In particular, the present invention is based on the discovery that the administration of an antibody that specifically binds CD56 linked to a cytotoxic compound (e.g., an immunoconjugate) in combination with a chemotherapeutic agent selected from the group consisting of a gemcitabine compound, a topotecan compound, and a doxorubicin compound improves the therapeutic index in the treatment of ovarian cancer over and above the additive effects of the anticancer agents when used as single agents in a mouse/human (xenograft) model system. In one embodiment of the invention, combinations of an antibody that specifically binds CD56 linked to a cytotoxic compound (i.e., an “immunoconjugate”) plus additional chemotherapeutic agents have a synergistic effect in the ovarian cancer therapeutic index (compared to expected combined additive effects of the single compounds and agents alone).

In some embodiments of the present invention, an antibody that specifically binds CD56 linked to a cytotoxic compound (e.g., an immunoconjugate) and a chemotherapeutic agent selected from the group consisting of a gemcitabine compound, a topotecan compound, and a doxorubicin compound are administered to a subject to treat ovarian cancer. The ovarian cancer can be platinum-resistant or platinum-sensitive. The ovarian cancer can be new or recurrent. Thus, an antibody that specifically binds CD56 linked to a cytotoxic compound (e.g., an immunoconjugate) and a chemotherapeutic agent selected from the group consisting of a gemcitabine compound, a topotecan compound, and a doxorubicin compound can be administered to a patient that has previously received treatment for ovarian cancer (e.g., surgical treatment, maintenance treatment, radiation, hormone therapy, or chemotherapy).

In some embodiments of the present invention, an antibody that specifically binds CD56 linked to a cytotoxic compound (e.g., an immunoconjugate) and a chemotherapeutic agent selected from the group consisting of a gemcitabine compound, a topotecan compound, and a doxorubicin compound are administered to a subject to treat platinum-resistant ovarian, peritoneal, or fallopian tube cancer.

The present invention also provides methods of modulating the growth of selected cell populations, such as ovarian cancer cells, by administering a therapeutically effective amount of such combinations. Embodiments of the invention comprise methods of simultaneous and/or sequential administration of an immunoconjugate (e.g., antibodies linked to cytotoxic compounds) and a chemotherapeutic agent. In a preferred embodiment, methods of the invention comprise administration of an immunoconjugate (e.g., lorvotuzumab mertansine (also referred to as IMGN901)) in the form of a pharmaceutically acceptable composition and administration of one or more chemotherapeutic agents (e.g., a gemcitabine, a topotecan, or a doxorubicin compound) administered in the form of a separate pharmaceutically acceptable composition.

In one embodiment, an anti-CD56 antibody or immunoconjugate thereof (e.g., IMGN901) and a gemcitabine compound are administered. In another embodiment, an anti-CD56 antibody or immunoconjugate thereof (e.g., IMGN901), a gemcitabine compound, and at least one other chemotherapeutic agent are administered. In another embodiment, an anti-CD56 antibody or immunoconjugate thereof (e.g., IMGN901) and a topotecan compound are administered. In another embodiment, an anti-CD56 antibody or immunoconjugate thereof (e.g., IMGN901), a topotecan compound, and at least one other chemotherapeutic agent are administered. In another embodiment, an anti-CD56 antibody or immunoconjugate thereof (e.g., IMGN901) and a doxorubicin compound are administered. In another embodiment, an anti-CD56 antibody or immunoconjugate thereof (e.g., IMGN901), a doxorubicin compound, and at least one other chemotherapeutic agent are administered.

In one embodiment, an anti-CD56 antibody or immunoconjugate thereof (e.g., IMGN901), a gemcitabine compound and a topotecan compound are administered. In another embodiment, an anti-CD56 antibody or immunoconjugate thereof (e.g., IMGN901), a gemcitabine compound, a topotecan compound, and at least one other chemotherapeutic agent are administered. In another embodiment, an anti-CD56 antibody or immunoconjugate thereof (e.g., IMGN901), a gemcitabine compound, and a doxorubicin compound are administered. In another embodiment, an anti-CD56 antibody or immunoconjugate thereof (e.g., IMGN901) a gemcitabine compound, a doxorubicin compound, and at least one other chemotherapeutic agent are administered. In another embodiment, an anti-CD56 antibody or immunoconjugate thereof (e.g., IMGN901), a gemcitabine compound, a topotecan compound, and a doxorubicin compound are administered. In another embodiment, an anti-CD56 antibody or immunoconjugate thereof (e.g., IMGN901), a gemcitabine compound, a topotecan compound, a doxorubicin compound, and at least one other chemotherapeutic agent are administered.

In another embodiment, an anti-CD56 antibody or immunoconjugate thereof (e.g., IMGN901), at least one agent selected from the group consisting of a gemcitabine compound, a topotecan compound, and a doxorubicin compound, and at least one other chemotherapeutic agent are administered.

In one embodiment, an anti-CD56 antibody or immunoconjugate thereof (e.g., IMGN901) and at least one agent selected from the group consisting of a gemcitabine compound, a topotecan compound, and a doxorubicin compound, and optionally at least one other chemotherapeutic agent are administered sequentially (in any order). The anti-CD56 antibody or immunoconjugate thereof (e.g., IMGN901), gemcitabine compound, topotecan compound, doxorubicin compound, and/or other chemotherapeutic agent can be administered over a period of hours, days, weeks, or months. In another embodiment, an anti-CD56 antibody or immunoconjugate thereof (e.g., IMGN901) and at least one agent selected from the group consisting of a gemcitabine compound, a topotecan compound, and a doxorubicin compound, and optionally at least one other chemotherapeutic agent are administered simultaneously.

In one embodiment, pharmaceutical compositions of the invention comprise a humanized antibody N901-maytansinoid conjugate (e.g., huN901-DM1/lorvotuzumab mertansine/IMGN901) and a gemcitabine compound. In another embodiment, pharmaceutical compositions of the invention comprise a humanized antibody N901-maytansinoid conjugate (e.g., huN901-DM1/lorvotuzumab mertansine/IMGN901) and a topotecan compound. In a further embodiment, pharmaceutical compositions of the invention comprise a humanized antibody N901-maytansinoid conjugate (e.g., huN901-DM1/lorvotuzumab mertansine/IMGN901) and a doxorubicin compound.

In one embodiment, a pharmaceutical composition comprises an anti-CD56 antibody or immunoconjugate thereof (e.g., IMGN901) and a gemcitabine. In one embodiment, a pharmaceutical composition comprises an anti-CD56 antibody or immunoconjugate thereof (e.g., IMGN901), a gemcitabine, and at least one other chemotherapeutic agent. In one embodiment, a pharmaceutical composition comprises an anti-CD56 antibody or immunoconjugate thereof (e.g., IMGN901) and a topotecan. In one embodiment, a pharmaceutical composition comprises an anti-CD56 antibody or immunoconjugate thereof (e.g., IMGN901), a topotecan, and at least one other chemotherapeutic agent. In one embodiment, a pharmaceutical composition comprises an anti-CD56 antibody or immunoconjugate thereof (e.g., IMGN901) and a doxorubicin compound. In one embodiment, a pharmaceutical composition comprises an anti-CD56 antibody or immunoconjugate thereof (e.g., IMGN901), a doxorubicin compound, and at least one other chemotherapeutic agent.

In one embodiment, a pharmaceutical composition comprises an anti-CD56 antibody or immunoconjugate thereof (e.g., IMGN901), a gemcitabine compound and a topotecan compound. In one embodiment, a pharmaceutical composition comprises an anti-CD56 antibody or immunoconjugate thereof (e.g., IMGN901), a gemcitabine compound, a topotecan compound, and at least one other chemotherapeutic agent. In one embodiment, a pharmaceutical composition comprises an anti-CD56 antibody or immunoconjugate thereof (e.g., IMGN901), a gemcitabine compound, and a doxorubicin compound. In one embodiment, a pharmaceutical composition comprises an anti-CD56 antibody or immunoconjugate thereof (e.g., IMGN901), a gemcitabine compound, a doxorubicin compound, and at least one other chemotherapeutic agent. In one embodiment, a pharmaceutical composition comprises an anti-CD56 antibody or immunoconjugate thereof (e.g., IMGN901), a gemcitabine compound, a topotecan compound, and a doxorubicin compound. In one embodiment, a pharmaceutical composition comprises an anti-CD56 antibody or immunoconjugate thereof (e.g., IMGN901), a gemcitabine compound, a topotecan compound, a doxorubicin compound, and at least one other chemotherapeutic agent.

In one embodiment, a pharmaceutical composition comprises an anti-CD56 antibody or immunoconjugate thereof (e.g., IMGN901), at least one agent selected from the group consisting of a gemcitabine compound, a topotecan compound, and a doxorubicin compound, and at least one other chemotherapeutic agent.

In one embodiment, pharmaceutical compositions of the invention further comprise a pharmaceutically acceptable carrier.

In one embodiment, the immunoconjugate is a humanized antibody N901-maytansinoid conjugate (e.g., lorvotuzumab mertansine) administered in combination with a chemotherapeutic agent selected from the group consisting of a gemcitabine compound, a topotecan compound, and a doxorubicin, wherein the combination has therapeutic synergy or improves the therapeutic index in the treatment of ovarian cancer compared to the additive effects of using the immunoconjugate alone, or the chemotherapeutic agent alone.

In one embodiment, administration of an anti-CD56 antibody or immunoconjugate thereof (e.g., IMGN901) and at least one agent selected from the group consisting of a gemcitabine, a topotecan, and a doxorubicin compound can be a dose that is lower than the dose of the anti-CD56 antibody or immunoconjugate thereof (e.g., IMGN901), gemcitabine compound, topotecan compound, or doxorubicin compound that would be administered alone. In some embodiments, an anti-CD56 antibody or immunoconjugate thereof (e.g., IMGN901) and at least one agent selected from the group consisting of a gemcitabine, a topotecan, and a doxorubicin compound is administered to a patient in need thereof, and the dose of the antibody or immunoconjugate is 1 to 150 mg/m², (e.g., 30 mg/m², 36 mg/m², 48 mg/m², 60 mg/m², 75 mg/m², 90 mg/m², or 112 mg/m²), the dose of the gemcitabine compound is equal to or less than 1000 mg/m² (e.g., 900 mg/m², 800 mg/m², 750 mg/m², 700 mg/m², 600 mg/m², 500 mg/m², or 300 mg/m²), the dose of the topotecan compound is equal to or less than 1.5 mg/m² (e.g., 1 mg/m², 0.5 mg/m², or 0.25 mg/m²), the dose of the doxorubicin compound is equal to or less than 75 mg/m² (e.g., 70 mg/m², 65 mg/m², 60 mg/m², 50 Mg/m², 45 mg/m², 30 mg/m², or 25 mg/m²), or a combination thereof.

“Therapeutic synergy,” as used herein, means that a combination of a conjugate and one or more chemotherapeutic agent(s) produces a therapeutic effect in ovarian cancer treatment which is greater than the additive effects of a conjugate and chemotherapeutic agents when each are used alone.

These and other aspects of the present invention are described in detail herein.

### BRIEF DESCRIPTION OF THE DRAWINGS/FIGURES

FIG. 1: Shows the anti-tumor effect of lorvotuzumab mertansine (IMGN901) and gemcitabine in established subcutaneous COLO 720E human ovarian carcinoma xenografts.

FIG. 2: Shows the anti-tumor effect of lorvotuzumab mertansine (IMGN901) and topotecan in established subcutaneous COLO 720E human ovarian carcinoma xenografts.

FIG. 3: Shows the anti-tumor effect of lorvotuzumab mertansine (IMGN901) and DOXIL® in established subcutaneous COLO 720E human ovarian carcinoma xenografts.

FIG. 4: Shows the sequences of huN901.

### DETAILED DESCRIPTION OF THE INVENTION

### Ovarian, Peritoneal, and Fallopian Tube Cancer

Ovarian cancer is a cancerous growth arising from different parts of the ovary. The most common form of ovarian cancer (>80%) arises from the outer lining (epithelium) of the ovary. However, the fallopian tube (epithelium) is also prone to develop into the same kind of cancer as seen in the ovaries. Since the ovaries and tubes are closely related to each other, it is hypothesized that these cells can mimic ovarian cancer. Other forms of ovarian cancer can arise from egg cells (i.e., a germ cell tumor). The risk of ovarian cancer increases with age and decreases with pregnancy. Lifetime risk has been estimated at about 1.6%, but women with affected first-degree relatives have a higher (˜5%) risk. Women with a mutated BRCA1 or BRCA2 gene carry a risk between 25% and 60% depending on the specific mutation. Ovarian cancer is the fifth leading cause of death from cancer in women and the leading cause of death from gynecological cancer. Fallopian tube cancer or tubal cancer is relatively rare and currently is believed to account for 1-2% of gynecological cancers. Tubal cancer is most typically an adenocarcinoma and, because of its location, it is sometimes mistaken for ovarian cancer. Peritoneal cancer is a cancer of the cells lining the peritoneum, or abdominal cavity. Some studies indicate that up to 15% of serous ovarian cancers are actually primary peritoneal carcinomas in origin.

The present invention provides improved pharmaceutical compositions and methods for use in the treatment of ovarian, peritoneal, and fallopian tube cancer. The ovarian, peritoneal, or fallopian tube cancer can be platinum-resistant or platinum-sensitive. The ovarian cancer can be, for example, advanced ovarian cancer, refractory ovarian cancer, or recurrent ovarian cancer.

The ovarian, peritoneal, or fallopian tube cancer can be a new cancer or a recurrent or relapsed cancer. Thus, in some embodiments, an anti-CD56 antibody or immunoconjugate thereof (e.g., IMGN901) and a topotecan compound, a gemcitabine compound, and/or a doxorubicin compound are administered to a patient with a new cancer. In other embodiments, an anti-CD56 antibody or immunoconjugate thereof (e.g., IMGN901) and a topotecan compound, a gemcitabine compound, and/or a doxorubicin compound are administered to a patient that has previously received a treatment (e.g., surgical treatment, maintenance treatment, radiation, hormone therapy, or chemotherapy). For example, an anti-CD56 antibody or immunoconjugate thereof (e.g., IMGN901) and a topotecan compound, a gemcitabine compound, and/or a doxorubicin compound can be administered to a patient that previously received treatment with a topotecan compound, a gemcitabine compound, a doxorubicin compound, a platinum compound, or a combination thereof. The previous treatment can be a treatment that was successful or unsuccessful. For example, an anti-CD56 antibody or immunoconjugate thereof (e.g., IMGN901) and a topotecan compound, a gemcitabine compound, and/or a doxorubicin compound can be administered to a subject in need thereof that has previously been treated for ovarian, peritoneal, or fallopian tube cancer with a platinum compound, wherein the ovarian, peritoneal, or fallopian tube cancer was platinum-resistant or platinum-sensitive.

### Conjugates and Immunoconjugates

One component of the present invention utilizes a CD56 antibody linked or “conjugated” to a cytotoxic compound (e.g., a maytansinoid compound such as DM1 (described further below)) to produce a “conjugate.” Thus, when the CD56 antibody (or an antigen-binding fragment thereof; such as a fragment containing the antigen-binding domain of a CD56 antibody) is linked to a cytotoxic compound, this combined antibody/cytotoxic compound moiety is referred to herein as an “immunoconjugate.” Immunoconjugates of the present invention are combined with additional cytotoxic compounds or chemotherapeutic agents to produce synergistic effects (synergy) useful in the treatment of ovarian, peritoneal, or fallopian tube cancer.

In one embodiment, the average molar ratio of the cytotoxic agent (e.g., maytansinoid) to the cell-binding agent in the cell-binding agent cytotoxic agent conjugate is about 1 to about 10. The terms “MAR,” “Maytansinoid-Ab Ratio,” “drug load,” “DAR,” and “Drug-Ab Ratio” can be used herein to characterize the ratio of cytotoxic agent to cell-binding agent in a conjugate comprising a maytansinoid compound as the cytotoxic agent and an antibody or fragment thereof as the cell binding agent. Thus, in some embodiments, the MAR is about 1 to about 10, about 2 to about 7, about 3 to about 5, about 2.5 to about 4.5 (e.g., about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5), about 3.0 to about 4.0, about 3.2 to about 4.2, about 4.5 to 5.5 (e.g., about 4.5, about 4.6, about 4.7; about 4.8, about 4.9, about 5.0, about 5.1, about 5.2, about 5.3, about 5.4, about 5.5). In one aspect, the number of drug molecules that can be attached to a cell binding agent can average from about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1). In some embodiments, the drug is N^{2′}-deacetyl-N^{2′}-(3-mercapto-1-oxopropyl)-maytansine (DM1).

Thus, in one aspect, an immunocongugate comprises 1 maytansinoid per antibody. In another aspect, an immunocongugate comprises 2 maytansinoids per antibody. In another aspect, an immunocongugate comprises 3 maytansinoids per antibody. In another aspect, an immunocongugate comprises 4 maytansinoids per antibody. In another aspect, an immunocongugate comprises 5 maytansinoids per antibody. In another aspect, an immunocongugate comprises 6 maytansinoids per antibody. In another aspect, an immunocongugate comprises 7 maytansinoids per antibody. In another aspect, an immunocongugate comprises 8 maytansinoids per antibody.

In one aspect, an immunoconjugate comprises about 1 to about 8 maytansinoids per antibody. In another aspect, an immunoconjugate comprises about 2 to about 7 maytansinoids per antibody. In another aspect, an immunoconjugate comprises about 2 to about 6 maytansinoids per antibody. In another aspect, an immunoconjugate comprises about 2 to about 5 maytansinoids per antibody. In another aspect, an immunoconjugate comprises about 3 to about 5 maytansinoids per antibody. In another aspect, an immunoconjugate comprises about 3 to about 4 maytansinoids per antibody.

In one aspect, a composition comprising immunoconjugates has an average of about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1) drug molecules (e.g., maytansinoids) attached per antibody. In one aspect, a composition comprising immunoconjugates has an average of about 1 to about 8 drug molecules (e.g., maytansinoids) per antibody. In one aspect, a composition comprising immunoconjugates has an average of about 2 to about 7 drug molecules (e.g., maytansinoids) per antibody. In one aspect, a composition comprising immunoconjugates has an average of about 2 to about 6 drug molecules (e.g., maytansinoids) per antibody. In one aspect, a composition comprising immunoconjugates has an average of about 2 to about 5 drug molecules (e.g., maytansinoids) per antibody. In one aspect, a composition comprising immunoconjugates has an average of about 3 to about 5 drug molecules (e.g., maytansinoids) per antibody. In one aspect, a composition comprising immunoconjugates has an average of about 3 to about 4 drug molecules (e.g., maytansinoids) per antibody.

In one aspect, a composition comprising immunoconjugates has an average of about 2±0.5, about 3±0.5, about 4±0.5, about 5±0.5, about 6±0.5, about 7±0.5, or about 8±0.5 drug molecules (e.g., maytansinoids) attached per antibody. In one aspect, a composition comprising immunoconjugates has an average of about 3.5±0.5 drug molecules (e.g., maytansinoids) per antibody.

### Synergy

Chou and Talalay (Adv. Enzyme Regul., 22:27-55 (1984)) developed a mathematical method to describe the experimental findings of combined drug effects in a qualitative and quantitative manner. For mutually exclusive drugs, they showed that the generalized isobol equation applies for any degree of effect (see page 52 in Chou and Talalay). An isobol or isobologram is the graphic representation of all dose combinations of two drugs that have the same degree of effect, for example combinations of two cytotoxic drugs will affect the same degree of cell kill, such as 20% or 50% of cell kill. In isobolograms, straight line indicates additive effects, a concave curve (curve below the straight line) represents synergistic effects, and a convex curve (curve above the straight line) represents antagonistic effects. These curves also show that a combination of two mutually exclusive drugs will show the same type of effect over the whole concentration range, either the combination is additive, synergistic, or antagonistic. Most drug combinations show an additive effect. In some instances however, the combinations show less or more than an additive effect. These combinations are called antagonistic or synergistic, respectively. A combination manifests therapeutic synergy if it is therapeutically superior to one or other of the constituents used at its optimum dose. See, T. H. Corbett et al., Cancer Treatment Reports, 66, 1187 (1982). Tallarida R J (J Pharmacol Exp Ther. 2001 September; 298 (3):865-72) also notes “Two drugs that produce overtly similar effects will sometimes produce exaggerated or diminished effects when used concurrently. A quantitative assessment is necessary to distinguish these cases from simply additive action.”

A synergistic effect may be measured using the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. In another embodiment, a synergistic effect is determined by using the fractional inhibitory concentration (FIC). This fractional value is determined by expressing the IC₅₀ of a drug acting in combination, as a function of the IC₅₀ of the drug acting alone. For two interacting drugs, the sum of the FTC value for each drug represents the measure of synergistic interaction. Where the FIC is less than 1, there is synergy between the two drugs. An FIC value of 1 indicates an additive effect. The smaller the FIC value, the greater the synergistic interaction.

The term “IC₅₀” indicates the “half maximal inhibitory concentration.” IC₅₀ is a measure of the effectiveness of a compound at inhibiting a biological or a biochemical function or activity. This quantitative measure indicates the concentration of a particular drug or other substance that is required to inhibit a biological or biochemical function or activity to a degree which is one-half of the maximum activity. In one representation, IC₅₀ is the concentration of a substance which is required for 50% activity inhibition in vitro.

In one embodiment of the invention, it has been discovered that administration of a combination of a CD56-binding immunoconjugate, and a chemotherapeutic agent selected from the group consisting of a gemcitabine compound, a topotecan compound and a doxorubicin compound produce a synergistic therapeutic effect in the treatment of ovarian cancer.

Embodiments of the invention comprise methods of simultaneous and/or sequential administration (in any order) of an immunoconjugate (e.g., antibodies linked to cytotoxic compounds) and a chemotherapeutic agent. In a preferred embodiment, methods of the invention comprise administration of an immunoconjugate (e.g., lorvotuzumab mertansine or other immunoconjugates described herein) in the form of a pharmaceutically acceptable composition and administration of a chemotherapeutic agent (e.g., a gemcitabine, a topotecan, a doxorubicin compound or other chemotherapeutic agent) administered in the form of a separate pharmaceutically acceptable composition, sequentially (in any order). Embodiments of the invention further include repeat administration of immunoconjugates and chemotherapeutic agents at any therapeutically effective or acceptable interval.

The term “synergistic effect”, as used herein, refers to a greater-than-additive therapeutic effect produced by a combination of compounds wherein the therapeutic effect obtained with the combination exceeds the additive effects that would otherwise result from individual administration the compounds alone. Embodiments of the invention include methods or combinations of producing a synergistic effect in the treatment of ovarian cancer, wherein said effect is at least 5%, at least 10%, at least 20%, at least 30%, at least 40%, at least 50%, at least 60%, at least 70%, at least 80%, at least 90%, at least 100%, at least 200%, at least 500%, or at least 1000% greater than the corresponding additive effect.

In some embodiments, a synergistic effect is obtained in the treatment of ovarian cancer wherein one or more of the agents or compounds are administered in a “low dose” (i.e., using a dose or doses which would be considered non-therapeutic if administered alone), wherein the administration of the low dose compound or agent in combination with other compounds or agents (administered at either a low or therapeutic dose) results in a synergistic effect which exceeds the additive effects that would otherwise result from individual administration the compounds, at the equivalent doses, alone. In some embodiments, the synergistic effect is achieved via administration of one or more of the agents or compounds administered in a “low dose” wherein the low dose is provided to reduce or avoid toxicity or other undesirable side effects.

In one embodiment, a synergistic effect is obtained in the treatment of ovarian cancer wherein one or more of the agents or compounds administered in a low dose comprise any one of, or any combination of one or more of, lorvotuzumab mertansine (IMGN901), and/or a chemotherapeutic agent selected from the group consisting of a gemcitabine compound, a topotecan compound and a doxorubicin compound. In another embodiment, a synergistic effect is obtained in the treatment of ovarian cancer wherein the agents or compounds administered comprise low dose lorvotuzumab mertansine, and low dose chemotherapeutic agent, wherein the chemotherapeutic agent is selected from the group consisting of a gemcitabine compound, a topotecan compound and a doxorubicin compound.

### CD56 Antibodies and Fragments Thereof

Antibodies that specifically bind CD56 (i.e., “CD56 antibodies”) used in the present invention include any type of CD56 antibody or CD56-binding fragments, portions, or other antigen binding forms thereof. These include, for example, but without limitation various forms of antibodies and fragments thereof such as:
Antibodies and derivatives or analogues thereof such as:
   polyclonal or monoclonal antibodies or antigen-binding fragments thereof;
   chimeric, primatized, humanized, fully human antibodies or antigen-binding fragments thereof;
   resurfaced antibodies or antigen-binding fragments thereof (see, e.g., U.S. Pat. No. 5,639,641);
   epitope binding fragments of antibodies such as single-chain, r v, sFv, scFv, Fab, Fab′, and F(ab′)2 (Parham, J. Immunol. 131:2895-2902 (1983); Spring et al, J. Immunol. 113:470-478 (1974); Nisonoff et al, Arch. Biochem. Biophys. 89:230-244 (1960)).

Additional examples of the broad variety and nature of types of antigen binding molecules that may be generated and used as CD56-binding agents are discussed in further detail subsequently herein.

### Lorvotuzumab Mertansine (IMGN901)

The antibody portion of lorvotuzumab mertansine (a.k.a., IMGN901) was originally derived from N901. N901 is an IgG1 murine monoclonal antibody (also called anti-A N901) that is reactive with CD56, which is expressed on tumors of neuroendocrine origin. See e.g., Griffin et al, J. Immunol. 130:2947-2951 (1983) and U.S. Pat. No. 5,639,641.

The CD56 antigen is a neural cell adhesion molecule (NCAM) that is expressed on the surface of tumor cells of neuroendocrine origin, including small cell lung carcinomas (SCLC), carcinoid tumors and Merkel cell carcinomas (MCC). CD56 is expressed on approximately 56% of ovarian tumors. See, e.g., Whiteman, K. R. et. al., AACR Annual Meeting, Abstract No. 2135, “Preclinical Evaluation of IMGN901 (huN901-DM1) as a Potential Therapeutic for Ovarian Cancer” (April 2008). CD56 is also expressed on approximately 70% of multiple myelomas. See, e.g., Tassone, P. et al., Cancer Res. 64:4629-4636 (2004).

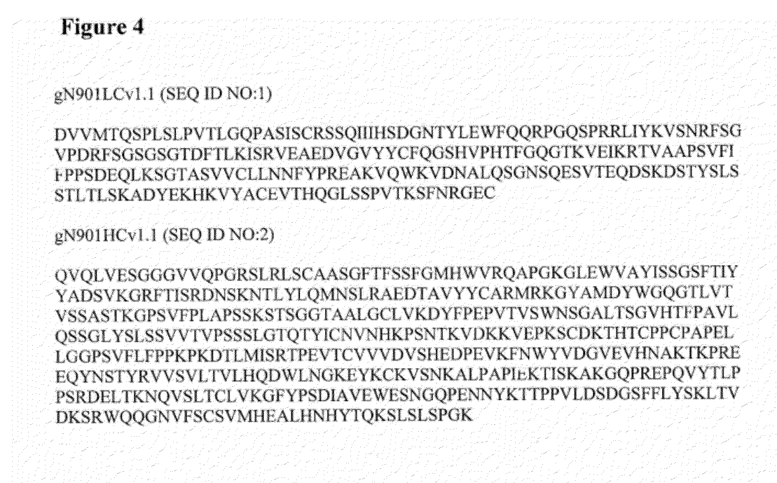

The preparation of different versions of humanized N901, is described, for example, by Roguska et al, Proc. Natl. Acad. Sci. USA, 91:969-973 (1994), and Roguska et al, Protein Eng., 9:895:904 (1996), the disclosures of which are incorporated by reference herein in their entirety. To denote a humanized antibody, the letters “hu” or “h” appear before the name of the antibody. For example, humanized N901 may be referred to as huN901 or hN901. The sequences for huN901 are provided in FIG. 4 as SEQ ID NOs: 1 and 2.

Lorvotuzumab mertansine (IMGN901) is an antibody-drug conjugate (ADC) comprised of the CD56-binding monoclonal antibody, huN901 (CDR grafted), the N-succinimidyl 4-(2-pyridyldithio)pentanoate (SPP) linker, and the maytansinoid cytotoxic agent, DM1. See, U.S. Pat. No. 7,303,749, Example 1, for an exemplary description of huN901/DM1 conjugation. The entirety of U.S. Pat. No. 7,303,749 (Inventor: R. V. J. Chari; Issued Dec. 4, 2007) is incorporated by reference herein. Additional information regarding maytansinoid compounds is also discussed further herein.

Lorvotuzumab mertansine (IMGN901) binds with high affinity to CD56 expressed on the surface of tumor cells. Once bound, the conjugate is internalized and the DM1 is released.

DM1 is an antimitotic agent that disrupts tubulin polymerization and microtubule assembly. See, Remillard S. et al., 1975, Science 189:1002-1005). See also, U.S. Pat. No. 7,303,749, Example 1, describing that “Ansamitocin P-3, provided by Takeda (Osaka, Japan) was converted to the disulfide-containing maytansinoid DM1, as described herein and in U.S. Pat. No. 5,208,020.” The entirety of U.S. Pat. No. 5,208,020 (Inventors: Chari et al.; Issued May 4, 1993) is incorporated by reference herein.

Lorvotuzumab mertansine (IMGN901) shows marked antitumor activity as a single agent in human xenograft preclinical models for ovarian cancer.

### Maytansinoids and Other Anti-Mitotic Agents

A mitotic inhibitor (anti-mitotic agent) is a type of drug commonly derived from natural substances such as plant alkaloids which are often used in cancer treatment and cytogenetic research. Cancer cells grow, and eventually metastasize, through continuous mitotic division. Generally, mitotic inhibitors prevent cells from undergoing mitosis by disrupting microtubule polymerization, thus preventing cancerous growth. Mitotic inhibitors work by interfering with and halting mitosis (usually during the M phase of the cell cycle), so that a cell can no longer divide. Polymerization of tubulin, which is necessary for mitosis to occur, may be suppressed by mitotic inhibitors, thereby preventing mitosis. Some examples of mitotic inhibitors used in the treatment of cancer include maytansanoids (e.g., DM1), paclitaxel, docetaxel, vinblastine, vincristine, and vinorelbine.

Maytansinoids that can be used in the present invention are well known in the art and can be isolated from natural sources according to known methods or prepared synthetically according to known methods. Examples of suitable maytansinoids include maytansinol and maytansinol analogues. Examples of suitable maytansinol analogues include those having a modified aromatic ring and those having modifications at other positions.

Some specific examples of suitable analogues of maytansinol having a modified aromatic ring include:
(1) C-19-dechloro (U.S. Pat. No. 4,256,746) (prepared by LAH reduction of ansamitocin P2);
(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and
(3) C-20-demethoxy, C-20-acyloxy (—OCOR), +/−dechloro (U.S. Pat. No. 4,294,757) (prepared by acylation using acyl chlorides).

Some specific examples of suitable analogues of maytansinol having modifications of other positions include:
(1) C-9-SH (U.S. Pat. No. 4,424,219) (prepared by the reaction of maytansinol with H₂S or P₂Sₛ);
(2) C-14-alkoxymethyl (demethoxy/CH₂OR) (U.S. Pat. No. 4,331,598);
(3) C-14-hydroxymethyl or acyloxymethyl (CH₂OH or CH₂OAc) (U.S. Pat. No. 4,450,254) (prepared from Nocardia);
(4) C-15-hydroxy/acyloxy (U.S. Pat. No. 4,364,866) (prepared by the conversion of maytansinol by Streptomyces);
(5) C-15-methoxy (U.S. Pat. Nos. 4,313,946 and 4,315,929) (isolated from Trewia nudiflora);
(6) C-18-N-demethyl (U.S. Pat. Nos. 4,362,663 and 4,322,348) (prepared by the demethylation of maytansinol by Streptomyces); and
(7) 4,5-deoxy (U.S. Pat. No. 4,371,533) (prepared by the titanium trichloride/LAH reduction of maytansinol).

A synthesis of thiol-containing maytansinoids useful in the present invention is disclosed in U.S. Pat. Nos. 5,208,020; 5,416,064; 6,333,410; 7,276,497; and 7,301,019.

Maytansinoids with a thiol moiety at the C-3 position, the C-14 position, the C-15 position or the C-20 position are all expected to be useful. The C-3 position is preferred and the C-3 position of maytansinol is especially preferred. Also preferred are an N-methyl-alanine-containing C-3 thiol moiety maytansinoid, and an N-methyl-cysteine-containing C-3 thiol moiety maytansinoid, and analogues of each.

Some specific examples of N-methyl-alanine-containing C-3 thiol moiety maytansinoid derivatives useful in the present invention are represented by the formula M1, M2, M3, M6 and M7.

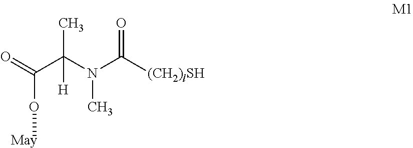

wherein:
l is an integer of from 1 to 10; and
may is a maytansinoid.

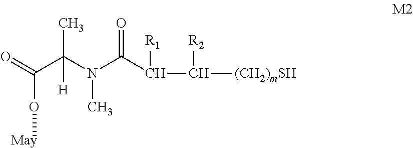

wherein:
R₁ and R₂ are H, CH₃ or CH₂CH₃, and may be the same or different;
m is 0, 1, 2 or 3; and
may is a maytansinoid.

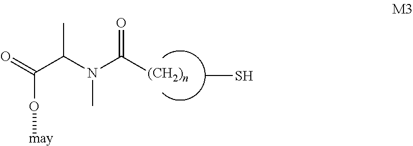

wherein:
n is an integer of from 3 to 8; and
may is a maytansinoid.

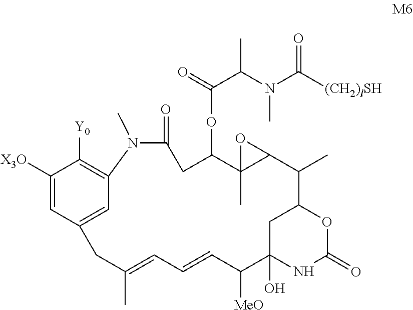

wherein:
l is 1, 2 or 3;

### Y₀ is Cl or H; and

### X₃ is H or CH₃.

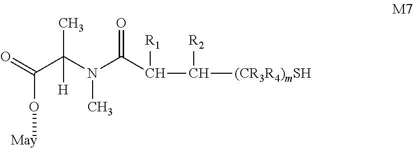

wherein:
R₁, R₂, R₃, R₄ are H, CH₃ or CH₂CH₃, and may be the same or different;
m is 0, 1, 2 or 3; and
may is a maytansinoid.

Some specific examples of N-methyl-cysteine-containing C-3 thiol moiety maytansinoid derivatives useful in the present invention are represented by the formula M4 and M5.

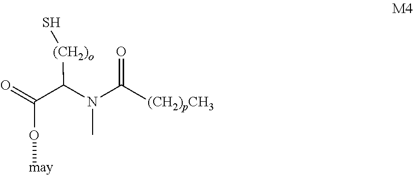

wherein:
o is 1, 2 or 3;
p is an integer of 0 to 10; and
may is a maytansinoid.

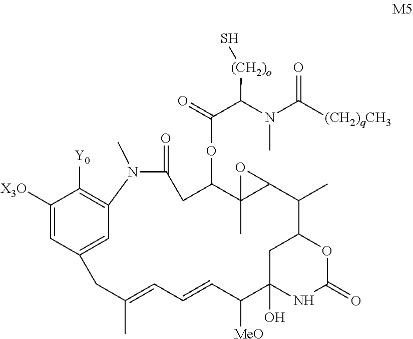

wherein:
o is 1, 2 or 3;
q is an integer of from 0 to 10;
Y₀ is Cl or H; and
X₃ is H or CH₃.

A further specific example of a maytansinoid compound useful in the present invention, DM1, is represented by the formula M8.

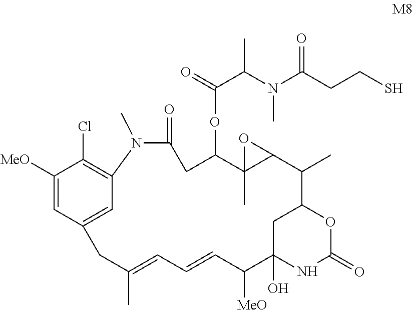

Some embodiments of maytansinoids are also described in U.S. Pat. Nos. 5,208,020; 5,416,064; 6,333,410; 6,441,163; 6,716,821; RE39,151; and 7,276,497.

In one embodiment of the invention a pharmaceutical composition used in the treatment of ovarian cancer comprises lorvotuzumab mertansine (IMGN901), and a chemotherapeutic agent selected from the group consisting of a gemcitabine compound, a topotecan compound and a doxorubicin compound. In one embodiment, pharmaceutical compositions of the invention comprise lorvotuzumab mertansine (IMGN901) and a gemcitabine compound. In another embodiment, pharmaceutical compositions of the invention comprise lorvotuzumab mertansine (IMGN901) and a topotecan compound. In a further embodiment, pharmaceutical compositions of the invention comprise lorvotuzumab mertansine (IMGN901) and a doxorubicin compound.

### Conjugate Linkage

A cell-binding agent of the invention may be modified by reacting a bifunctional crosslinking reagent with the cell-binding agent, thereby resulting in the covalent attachment of a linker molecule to the cell-binding agent. As used herein, a “bifunctional crosslinking reagent” is any chemical moiety that covalently links a cell-binding agent to a drug, such as the drugs described herein. In a preferred embodiment of the invention, a portion of the linking moiety is provided by the drug. In this respect, the drug comprises a linking moiety that is part of a larger linker molecule that is used to join the cell-binding agent to the drug. For example, to form the maytansinoid DM1 or DM4, the ester side chain at the C-3 position of maytansine is modified to have a free sulfhydryl group (SH), as described in U.S. Pat. Nos. 5,208,020; 6,333,410; and 7,276,497. This thiolated form of maytansine can react with a modified cell-binding agent to form a conjugate. Therefore, the final linker is assembled from two components, one of which is provided by the crosslinking reagent, while the other is provided by the side chain from DM1 or DM4.

Any suitable bifunctional crosslinking reagent can be used in connection with the invention, so long as the linker reagent provides for retention of the therapeutic (e.g., cytotoxicity), and targeting characteristics of the drug and the cell-binding agent, respectively. Preferably, the linker molecule joins the drug to the cell-binding agent through chemical bonds (as described above), such that the drug and the cell-binding agent are chemically coupled (e.g., covalently bonded) to each other. Preferably, the linking reagent is a cleavable linker. More preferably, the linker is cleaved under mild conditions, i.e., conditions within a cell under which the activity of the drug is not affected. Examples of suitable cleavable linkers include disulfide linkers, acid labile linkers, photolabile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Photo labile linkers are useful at the body surface and in many body cavities that are accessible to light. Furthermore, infrared light can penetrate tissue. Peptidase labile linkers can be used to cleave certain peptides inside or outside cells (see e.g., Trouet et al., Proc. Natl. Acad. Sci. USA, 79: 626-629 (1982), and Umemoto et al., Int. J. Cancer, 43: 677-684 (1989)).

In one embodiment, a cytotoxic compound is linked to a cell-binding agent through a disulfide bond or a thioether bond. The linker molecule comprises a reactive chemical group that can react with the cell-binding agent. Exemplary reactive chemical groups for reaction with the cell-binding agent are N-succinimidyl esters and N-sulfosuccinimidyl esters. Additionally the linker molecule may comprise a reactive chemical group, such as a dithiopyridyl group that can react with the drug to form a disulfide bond. Particular embodiments of linker molecules include, for example, N-succinimidyl 3-(2-pyridyldithio)propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), and other reactive cross-linkers which are described in U.S. Pat. No. 6,913,748. In one embodiment, a cytotoxic compound is linked to a cell-binding agent using a linker molecule comprising N-succinimidyl 3-(2-pyridyldithio)propionate (SPDP). In another embodiment, a cytotoxic compound is linked to a cell-binding agent using a linker molecule comprising N-succinimidyl 4-(2-pyridyldithio)butanoate (SPDB). In a another embodiment, a cytotoxic compound is linked to a cell-binding agent using a linker molecule comprising N-succinimidyl 4-(2-pyridyldithio)pentanoate (SPP).

Embodiments of the invention include both cleavable linkers and non-cleavable linker to generate the above-described conjugate. A non-cleavable linker is any chemical moiety that is capable of linking a drug, such as a maytansinoid, a Vinca alkaloid, a dolastatin, an auristatin, or a cryptophycin, to a cell-binding agent in a stable, covalent manner. Non-cleavable linkers are substantially resistant to acid-induced cleavage, light-induced cleavage, peptidase-induced cleavage, esterase-induced cleavage, and disulfide bond cleavage, at conditions under which the drug or the cell-binding agent remains active.

Suitable crosslinking reagents that form non-cleavable linkers between a drug and the cell-binding agent are well known in the art. Examples of non-cleavable linkers include linkers having an N-succinimidyl ester or N-sulfosuccinimidyl ester moiety for reaction with the cell-binding agent, as well as a maleimido- or haloacetyl-based moiety for reaction with the drug. Crosslinking reagents comprising a maleimido-based moiety include N-succinimidyl 4-(maleimidomethyl)cyclohexanecarboxylate (SMCC), N-succinimidyl-4-(N-maleimidomethyp-cyclohexane-1-carboxy-(6-amidocaproate), which is a “long chain” analog of SMCC (LC-SMCC), kappa-maleimidoundecanoic acid N-succinimidyl ester (KMUA), gamma-maleimidobutyric acid N-succinimidyl ester (GMBS), epsilon-maleimidocaproic acid N-hydroxysuccinimide ester (EMCS), m-maleimidobenzoyl-N-hydroxysuccinimide ester (MBS), N-(alpha-maleimidoacetoxy)-succinimide ester (AMAS), succinimidyl-6-(beta-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP).

Other crosslinking reagents lacking a sulfur atom that form non-cleavable linkers may also be used as embodiments of the invention. Such linkers can be derived, for example, from dicarboxylic acid based moieties. Suitable dicarboxylic acid based moieties include, but are not limited to, am-dicarboxylic acids of the general formula (IX):

HOOC—X₁—Yₙ—Zₘ—COOH (IX),

wherein X is a linear or branched alkyl, alkenyl, or alkynyl group having 2 to 20 carbon atoms, Y is a cycloalkyl or cycloalkenyl group bearing 3 to 10 carbon atoms, Z is a substituted or unsubstituted aromatic group bearing 6 to 10 carbon atoms, or a substituted or unsubstituted heterocyclic group wherein the hetero atom is selected from N, O or S, and wherein l, m, and n are each 0 or 1, provided that l, m, and n are all not zero at the same time.

Exemplary non-cleavable linkers disclosed herein are described in U.S. patent application Ser. No. 10/960,602 (U.S. Publication No. 2005/0169933). Other linkers which can be used in the present invention include charged linkers or hydrophilic linkers and are described in U.S. patent application Ser. Nos., 12/433,604 (U.S. Publication No. 2009/0274713) and Ser. No. 12/574,466 (U.S. Publication No. 2010/0129314), respectively.

Alternatively, as disclosed in U.S. Pat. No. 6,441,163 B1, the drug can be first modified to introduce a reactive ester suitable to react with a cell-binding agent. Reaction of these maytansinoids containing an activated linker moiety with a cell-binding agent provides another method of producing a cleavable or non-cleavable cell-binding agent maytansinoid conjugate.

### Synergistic Combinations of Lorvotuzumab Mertansine and a Chemotherapeutic Agent

Lorvotuzumab mertansine (IMGN901) has been shown to be highly active in combination with paclitaxel/carboplatin therapy in preclinical efficacy studies in ovarian xenograft models. The maytansinoid immunoconjugate lorvotuzumab mertansine has anti-mitotic activity through inhibition of tubulin polymerization. Combination of lorvotuzumab mertansine with therapeutic agents with different mechanisms of action may result in highly effective therapeutic synergy in the treatment of ovarian cancer. The favorable toxicity profile of lorvotuzumab mertansine and other maytansinoid immunoconjugates suggests that combinations with approved agents will be well tolerated, and it may be able to replace other more toxic agents in multi-drug combinations that are currently standard treatments. The therapeutic synergy of lorvituzumab mertansine in combination with a variety of ovarian cancer chemotherapeutics was evaluated in the COLO 720 xenograft model in athymic nude mice.

Gemcitabine (GEMZAR®), a nucleoside metabolic inhibitor, is used in the treatment of various carcinomas, for example, non-small cell lung cancer, pancreatic cancer, bladder cancer, breast cancer and refractory or resistant ovarian cancer. It is approved for use in combination with carboplatin, but has also been used in non-platinum containing regimens and has shown activity as a single agent in heavily pretreated patients. Treatment with lorvotuzumab mertansine resulted in only modest antitumor activity, while gemcitabine single-agent therapy was inactive against COLO 720E xenografts. Combination treatment of lorvotuzumab mertansine with gemcitabine was highly active, with partial regressions (PR) in 4/6 mice and complete regressions in 2/6 mice, compared with no regressions in either single-agent group.

Topotecan (HYCAMTIN®) is a topoisomerase I inhibitor which is used to treat platinum-resistant, platinum-refractory and metastatic ovarian cancers. Topotecan is approved for single-agent treatments, and is also used in combination with platinum agents. The combination of lorvotuzumab mertansine with topotecan was also highly active against COLO 720E xenografts, demonstrating synergistic activity in combination with complete regressions in all of the mice for the duration of the study (120 days) whereas there were no tumor-free mice in either single-agent group at the end of the study.

PEGylated liposomal doxorubicin (PLD, DOXIL®) is a therapeutic comprised of the anthracycline doxorubicin encapsulated in liposomes. DOXIL® is approved for the treatment of platinum/taxane-refractory ovarian cancers. Other doxorubicin compounds, including but not limited to, hydroxydaunorubicin, ADRIAMYCIN®, and RUBEX®, are also contemplated for use in the methods and compositions of the present invention. Single-agent treatment with lorvotuzumab mertansine or DOXIL® was active against COLO 720E xenografts, though there was only one CR in the lorvotuzumab mertansine treatment group. In combination, however, lorvotuzumab mertansine plus DOXIL® was highly active, with partial regressions in all of the mice (6/6) and CR in 2/6 mice.

All of the combination treatments described herein were well-tolerated, without evidence of increased toxicity in combination relative to single-agent therapy. The strong activity of these combinations resulted in synergistic responses, in terms of tumor regressions in combination versus single-agent groups.

### Dosing and Administration

Embodiments of the invention include immunoconjugates and cytotoxic compounds/chemotherapeutic agents used with pharmaceutically acceptable carriers, diluents, and/or excipients, which are well known, and can be determined, by one of skill in the art as the clinical situation warrants. Examples of suitable carriers, diluents and/or excipients include: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

Compounds and compositions described herein may be administered in appropriate forms and via routes such as would be used by one of skill in the art. Each composition can be formulated and administered in a different way. Some examples of various possible modes of administration for each composition include, without limitation, parenteral, intravenous, intraarterial, intraperitoneal, subcutaneous, intramuscular, intradermal. For various modes of administration, the compounds or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants. Compositions can also be in the form of sterile solid compositions which can be dissolved or dispersed in sterile water or any other injectable sterile medium.

Pharmaceutical compositions may be administered in any order or at any interval as determined by one of skill in the art. For example, but without limitation, a CD56-binding agent linked to a cytotoxic compound (such as lorvotuzumab mertansine), and a chemotherapeutic agent selected from the group consisting of a gemcitabine compound, a topotecan compound and a doxorubicin compound may be administered sequentially (in any order), simultaneously, or via any combination of sequential and simultaneous administrations. Any combination of sequential or simultaneous administration protocols may be used and implemented as decided and determined by one of skill in the art.

Administration of pharmaceutical compounds, whether simultaneous, sequential or a combination of both, may be performed according to any number of desired intervals of minutes (e.g., 0-60 minutes), hours (e.g., 0-24 hours), days (e.g., 0-7 days), and/or weeks (e.g., 0-52 weeks) as may be decided and determined by one of skill in the art. In one example, a pharmaceutical composition including IMGN901 is administered on a weekly basis. In another example, a pharmaceutical composition including IMGN901 is administered bi-weekly, once every three weeks, once every four weeks, or less often. The dosing can also vary over time, for example, starting with a once weekly dose for a period of time (e.g., for 1, 2, 3, 4, 5, or 6 weeks) followed by dosing once every two weeks, once every three weeks, once every four weeks, once every five weeks, or once every six weeks.

Administration of an anti-CD56 antibody or immunoconjugate thereof (e.g., IMGN901) and a topotecan compound, a gemcitabine compound, and/or a doxorubicin compound can result in the reduction of the required dosage of the anti-CD56 antibody or immunoconjugate thereof. In some embodiments, an anti-CD56 antibody or immunoconjugate thereof (e.g., IMGN901) and a topotecan compound, a gemcitabine compound, and/or a doxorubicin compound are administered to a subject in need thereof, and the dose of the antibody or immunoconjugate is lower than the dose of antibody or immunoconjugate that would be administered alone. Accordingly, in some embodiments, an anti-CD56 antibody or immunoconjugate thereof (e.g., IMGN901) and a topotecan compound, a gemcitabine compound, and/or a doxorubicin compound are administered and the antibody or immunoconjugate is administered at a dose of 1 to 150 mg/m², for example, 30 mg/m², 36 mg/m², 48 mg/m², 60 mg/m², 75 mg/m², 90 mg/m², 2 or 112 mg/m².

A “therapeutically effective amount” of the chemotherapeutic agents and immunoconjugates described herein refers to the dosage regimen for inhibiting the proliferation of selected cell populations and/or treating a patient's disease, and is selected in accordance with a variety of factors, including the age, weight, sex, diet and medical condition of the patient, the severity of the disease, the route of administration, and pharmacological considerations, such as the activity, efficacy, pharmacokinetic and toxicology profiles of the particular compound used. The “therapeutically effective amount” can also be determined by reference to standard medical texts, such as the Physicians Desk Reference 2011 (Publisher: PDR Network, LLC; ISBN 978-1-56363-780-3). Embodiments of the invention include methods of treating ovarian cancer in human and non-human mammals.

A gemcitabine compound can be administered, for example, at a dose of about 100-1500 mg/m², such as 1000 mg/m² or 1250 mg/m². Thus, in some embodiments, an anti-CD56 antibody or immunoconjugate thereof (e.g., IMGN901) and a gemcitabine compound are administered and the gemcitabine compound is administered at dose of 100-1500 mg/m², such as 1000 mg/m² or 1250 mg/m². In one example, a gemcitabine compound can be administered at a dose of 1000 mg/m² on days 1 and 8 of a 21-day course. Administration of an anti-CD56 antibody or immunoconjugate thereof (e.g., IMGN901) and a gemcitabine compound can result in the reduction of the required dosage of the gemcitabine compound. Thus, in some embodiments, an anti-CD56 antibody or immunoconjugate thereof (e.g., IMGN901) and a gemcitabine compound are administered to a subject in need thereof, and the dose of the gemcitabine compound is lower than the dose of gemcitabine compound that would be administered alone. Accordingly, in some embodiments, an anti-CD56 antibody or immunoconjugate thereof (e.g., IMGN901) and a gemcitabine compound are administered and the gemcitabine compound is administered at dose of less than 1000 mg/m², for example, 900 mg/m², 800 mg/m², 750 mg/m², 700 mg/m², 600 mg/m², 500 mg/m², or 300 mg/m².

A topotecan compound can be administered, for example, at a dose of about 1 to 10 mg/m², such as, 5 mg/m², 3 mg/m², 2 mg/m², 1.5 mg/m², 1 mg/m², 0.5 mg/m², or 0.25 mg/m². A topotecan compound can administered at a dose of 1.5 mg/m² for 5 consecutive days, starting on day 1 of a 21-day course. In some embodiments, an anti-CD56 antibody or immunoconjugate thereof (e.g., IMGN901) and a topotecan compound are administered and the topotecan compound is administered at dose of about 1 to 10 mg/m², such as, 5 mg/m², 3 mg/m², 2 mg/m², 1.5 mg/m², 1 mg/m², 0.5 mg/m², or 0.25 mg/m². Administration of an anti-CD56 antibody or immunoconjugate thereof (e.g., IMGN901) and a topotecan compound can result in the reduction of the required dosage of the topotecan compound. Thus, in some embodiments, an anti-CD56 antibody or immunoconjugate thereof (e.g., IMGN901) and a topotecan compound are administered to a subject in need thereof, and the dose of topotecan compound is lower than the dose of topotecan compound that would be administered alone. Accordingly, in some embodiments, an anti-CD56 antibody or immunoconjugate thereof (e.g., IMGN901) and a topotecan compound are administered and the topotecan compound is administered at dose of less than 1.5 mg/m², for example, 1 mg/m², 0.5 mg/m², or 0.25 mg/m². In another embodiment, an anti-CD56 antibody or immunoconjugate thereof (e.g., IMGN901) and a topotecan compound are administered and the topotecan compound is administered for a period of less than 5 days, for example, 4 days, 3 days, 2 days, or 1 day.

A doxorubicin compound can be administered, for example, at a dose of about 50 to 150 mg/m², such as, 60 mg/m² to 75 mg/m². A doxorubicin compound can administered at a dose of 60 mg/m² to 75 mg/m² on day 1 of a 21-day cycle. In some embodiments, an anti-CD56 antibody or immunoconjugate thereof (e.g., IMGN901) and a doxorubicin compound are administered and the doxorubicin compound is administered at dose of about 60 mg/m² to 75 mg/m². Administration of an anti-CD56 antibody or immunoconjugate thereof (e.g., IMGN901) and a platinum compound can result in the reduction of the required dosage of the doxorubicin compound. Thus, in some embodiments, an anti-CD56 antibody or immunoconjugate thereof (e.g., IMGN901) and doxorubicin compound are administered to a subject in need thereof, and the dose of the doxorubicin compound is lower than the dose of the doxorubicin compound that would be administered alone. Accordingly, in some embodiments, an anti-CD56 antibody or immunoconjugate thereof (e.g., IMGN901) and a doxorubicin compound are administered and the doxorubicin compound is administered at dose of less than 75 mg/m², for example, 65 mg/m², 60 mg/m², 55 mg/m², 50 mg/m², 45 mg/m², 30 mg/m², or mg/m². In another embodiment, an anti-CD56 antibody or immunoconjugate thereof (e.g., IMGN901) and a doxorubicin compound are administered and the doxorubicin compound is administered once every 28 days.

Examples of suitable protocols of administration of pharmaceutical/therapeutic compositions of the invention may be considered, without limitation, to include parameters such as follows. Pharmaceutical compositions may be given daily as an i.v. bolus, or as a continuous infusion.

Pharmaceutical compositions may be administered once a week for six weeks or longer. Pharmaceutical compositions may be administered once every two or three weeks. Bolus doses may be given in about 50 to about 400 ml of normal saline to which about 5 to about 10 ml of human serum albumin can be added. Continuous infusions may be given in about 250 to about 500 ml of normal saline, to which about 25 to about 50 ml of human serum albumin can be added, per 24 hour period. Dosages may be about 10 pg to about 1000 mg/kg per person, i.v. (range of about 100 ng to about 10 mg/kg).

About one to about four weeks after treatment, a patient may receive a second course of treatment. Specific clinical protocols with regard to route of administration, excipients, diluents, dosages, and times can be determined by the skilled artisan as the clinical situation warrants.

The present invention also provides pharmaceutical kits comprising one or more containers filled with one or more of the ingredients of the pharmaceutical compounds and/or compositions of the present invention, including, one or more immunoconjugates and one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products. Cancer therapies and their dosages, routes of administration and recommended usage are known in the art and have been described in such literature as the Physicians Desk Reference 2011 (Publisher: PDR Network, LLC; ISBN 978-1-56363-780-3) (“PDR”). The PDR discloses dosages of the agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician. The contents of the PDR are expressly incorporated herein in its entirety by reference. The contents of the PDR are expressly incorporated herein in their entirety by reference. One of skill in the art can review the PDR, using one or more of the following parameters, to determine dosing regimen and dosages of the chemotherapeutic agents and conjugates that can be used in accordance with the teachings of this invention. These parameters include:

| 1. | Comprehensive index | |
|---|---|---|
| | a) | by Manufacturer |
| | b) | Products (by company's or trademarked drug name) |
| | c) | Category index (for example, “nucleoside analogues,” |
| | | “antineoplastics: antimetabolites,” “antineoplastics: cytotoxic |
| | | agents,” “miscellaneous antineoplastics,” “antineoplastics: |
| | | antibiotics” etc.). |
| | d) | Generic/chemical index (non-trademark common drug names) |
| 2. | Color images of medications | |
| 3. | Product information, consistent with FDA labeling | |
| | a) | Chemical information |
| | b) | Function/action |
| | c) | Indications & Contraindications |
| | d) | Trial research, side effects, warnings |

### Analogues and Derivatives

One skilled in the art of therapeutic agents, such as cytotoxic agents or chemotherapeutic agents, will readily understand that each of the such agents described herein can be modified in such a manner that the resulting compound still retains the specificity and/or activity of the starting compound. The skilled artisan will also understand that many of these compounds can be used in place of the therapeutic agents described herein. Thus, the therapeutic agents of the present invention include analogues and derivatives of the compounds described herein.

### Immunoglobulins and Antibodies

The terms “antibody” and “immunoglobulin” may be used interchangeably herein. An antibody or immunoglobulin comprises at least the variable domain of a heavy chain, and normally comprises at least the variable domains of a heavy chain and a light chain. Basic immunoglobulin structures in vertebrate systems are well understood to those of ordinary skill in the art. See, e.g., Harlow et al., Antibodies: A Laboratory Manual, (Cold Spring Harbor Laboratory Press, 2nd ed. 1988).

The term “immunoglobulin” comprises various broad classes of polypeptides that can be distinguished biochemically. Those skilled in the art will appreciate that heavy chains are classified as gamma, mu, alpha, delta, or epsilon, with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernible to the skilled artisan in view of the instant disclosure and, accordingly, are within the scope of the instant invention. All immunoglobulin classes are clearly within the scope of the present invention. As one example, a typical IgG immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The N-terminal portion is a variable region and at the C-terminal portion is a constant region; the CH3 and CL domains actually comprise the carboxy-terminus of the heavy and light chain, respectively.

Variable regions allow the antibodies to selectively recognize and specifically bind epitopes on antigens. That is, the VL domain and VH domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers Casterman et al., Nature 363:446 448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to CD56 antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibodies or immunospecific fragments thereof of the present invention may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

The term “specifically binds,” generally means that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “1” may be deemed to have a higher specificity for a given epitope than antibody “2,” or antibody “1” may be said to bind to epitope “3” with a higher specificity than it has for related epitope “4.”

Monoclonal antibodies may be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hanimerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. For example, monoclonal antibodies can be prepared using CD56 knockout mice to increase the regions of epitope recognition. Monoclonal antibodies can be prepared using a wide variety of techniques known in the art including the use of hybridoma and recombinant and phage display technology as described elsewhere herein.

Antibody fragments that recognize specific epitopes may be generated by known techniques. For example, Fab and F(ab′)2 fragments may be produced recombinantly or by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

Those skilled in the art will also appreciate that DNA encoding antibodies or antibody fragments (e.g., antigen binding sites) may also be derived from antibody libraries, such as phage display libraries. In particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. Immunol. 24:952-958 (1994); Persic et al., Gene 187:9-18 (1997); Bitrton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties. Humanized antibodies are antibody molecules from a non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art, such as for example but without limitation including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741. Human antibodies can also be produced using transgenic mice which are incapable of expressing functional endogenous immunoglobulins, but which can express human immunoglobulin genes. See e.g., Lonberg and Huszar, Int. Rev. Immunol. 13:65-93 (1995); WO 98/24893; WO 96/34096; WO 96/33735; U.S. Pat. Nos. 5,413,923; 5,625,126; 5,633,425; 5,569,825; 5,661,016; 5,545,806; and 5,814,318.

Monoclonal antibody techniques allow for the production of specific cell-binding agents in the form of monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Sensitized human cells can also be used. Another method of creating monoclonal antibodies is the use of phage libraries of sFv (single chain variable region), specifically human sFv (see, e.g., Griffiths et al, U.S. Pat. No. 5,885,793; McCafferty et al, WO 92/01047; Liming et al, WO 99/06587.)

Selection of the appropriate cell-binding agent is a matter of choice that depends upon the particular cell population that is to be targeted, but in general monoclonal antibodies and epitope binding fragments thereof are preferred, if an appropriate one is available.

### Additional Guides to Methods and Techniques

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4th ed. Ed. Richard A. Goldsby, Thomas J. Kindt and Barbara A. Osborne, H. Freemand & Co. (2000); Roitt, I., Brostoff, J. and Male D., Immunology 6th ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

### EXAMPLES

The invention will now be described by reference to non-limiting examples.

Mice were inoculated with human ovarian cancer cell lines and allowed to become established (average tumor size of about 100 mm³) prior to treatment. Conjugate dosing is described based on antibody concentration. Efficacy is reported as both the % of tumor growth for treated vs. control (% T/C) and log cell kill (LCK) determined from the tumor doubling time and the tumor growth delay due to the treatment. Percent T/C values less than or equal to 42% and/or LCK values of 0.5 or greater are considered active; percent T/C values less than 10% are considered highly active (Bissery et al., Cancer Res, 51: 4845-4852 (1991).

### Example 1

### Anti-Tumor Effect of Combination Therapy of COLO 720E Human Ovarian Carcinoma Xenografts with Lorvotuzumab Mertansine and Gemcitabine

The anti-tumor effect of a combination of lorvotuzumab mertansine and gemcitabine was evaluated in an established subcutaneous xenograft model of ovarian cancer. Athymic nude mice were inoculated with COLO 720E human ovarian cancer cells (1×10⁷ cells/animal) injected subcutaneously into the right flank. When the tumors reached about 110 mm³ in size (10 days after tumor cell inoculation), the mice were randomly divided into four groups (six animals per group). A control group was treated with a single intravenous injection of PBS. A second group of mice were treated with the single agent lorvotuzumab mertansine at a dose of 11 mg/kg once weekly for three weeks (days 11, 18 and 25 post tumor cell inoculation) administered intravenously. The third group received single-agent gemcitabine administered intraperitoneally at a dose of 80 mg/kg every three days for a total of four injections (days 11, 14, 17 and 20 post tumor cell inoculation). A fourth group was treated with the combination of lorvotuzumab mertansine and gemcitabine with the same doses and routes of administration as for individual treatments. Tumor growth was monitored by measuring tumor size twice per week. Tumor size was calculated with the formula: length×width×height×½.

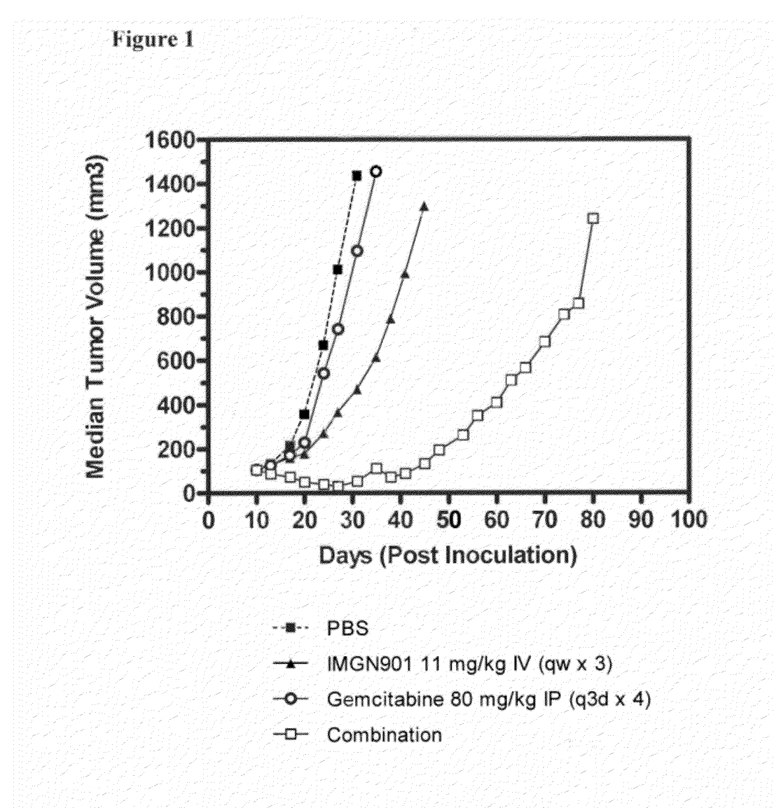

FIG. 1. Single-agent lorvotuzumab mertansine was active against COLO 720E xenografts, with a TIC of 38%, which is considered active by NCI standards. There were no tumor regressions observed. Single-agent gemcitabine treatment was inactive (T/C=77%). The combination of lorvotuzumab mertansine with gemcitabine was highly active by NCI standards (T/C=4%) with partial tumor regressions (PR) in four of six mice and complete regressions (CR) in two of six mice, with one mouse remaining tumor free until the end of the study (day 120).

### Example 2

### Anti-Tumor Effect of Combination Therapy of COLO 720E Human Ovarian Carcinoma Xenografts with Lorvotuzumab Mertansine and Topotecan

The anti-tumor effect of a combination of lorvotuzumab mertansine and topotecan was evaluated in an established subcutaneous xenograft model of ovarian cancer. Athymic nude mice were inoculated with COLO 720E human ovarian cancer cells (1×10⁷ cells/animal) injected subcutaneously into the right flank. When the tumors reached about 115 mm³ in size (12 days after tumor cell inoculation), the mice were randomly divided into four groups (six animals per group). A control group was treated with a single intravenous injection of PBS. A second group of mice were treated with the single agent lorvotuzumab mertansine at a dose of 11 mg/kg once weekly for three weeks (days 12, 19 and 26 post tumor cell inoculation) administered intravenously. The third group received single-agent topotecan administered intraperitoneally at a dose of 0.7 mg/kg on a 5/2/5 schedule (daily×5, 2 days off, daily×5; days 12-16 and 19-23 post tumor cell inoculation). A fourth group was treated with the combination of lorvotuzumab mertansine and topotecan with the same doses and routes of administration as for individual treatments. Tumor growth was monitored by measuring tumor size twice per week. Tumor size was calculated with the formula: length×width×height×½.

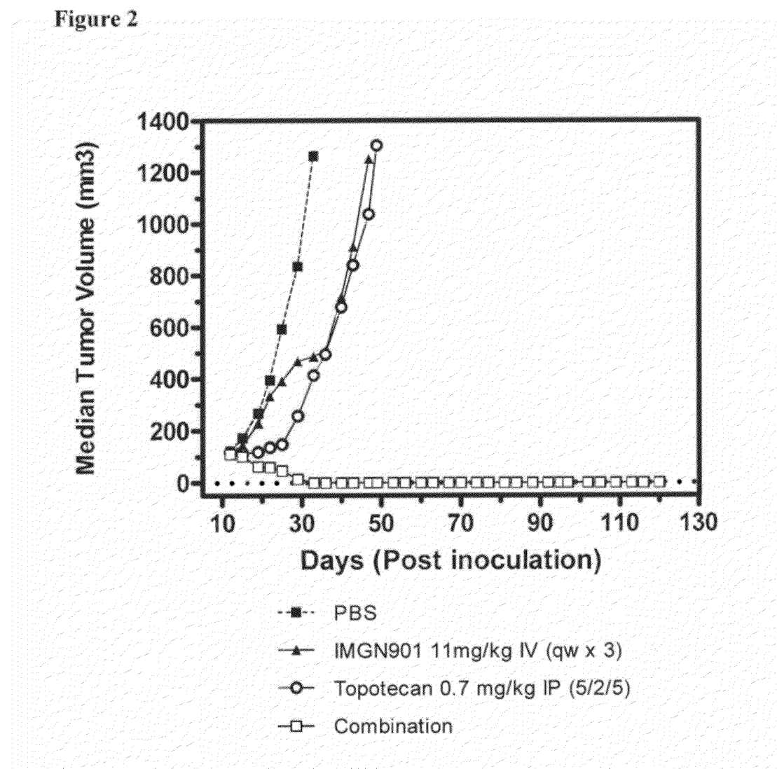

FIG. 2. Single-agent lorvotuzumab mertansine was inactive against COLO 720E xenografts in this study, with a T/C of 59%, though there was a partial regression (PR) in one of six mice. Single-agent topotecan treatment was active (T/C=26%) with a complete regression (CR) in one of six mice. The combination of lorvotuzumab mertansine with topotecan was highly active by NCl standards (T/C=6%) with CRs in all mice (six of six) which all remained tumor free until the end of the study (day 120).

### Example 3

### Anti-Tumor Effect of Combination Therapy of COLO 720E Human Ovarian Carcinoma Xenografts with Lorvotuzumab Mertansine and DOXIL®

The anti-tumor effect of a combination of lorvotuzumab mertansine and DOXIL® (i.e., PEGylated Liposomal Doxorubicin (PLD)) was evaluated in an established subcutaneous xenograft model of ovarian cancer. Athymic nude mice were inoculated with COLO 720E human ovarian cancer cells (1×10⁷ cells/animal) injected subcutaneously into the right flank. When the tumors reached about 100 mm³ in size (10 days after tumor cell inoculation), the mice were randomly divided into four groups (six animals per group). A control group was treated with a single intravenous injection of PBS. A second group of mice were treated with the single agent lorvotuzumab mertansine at a dose of 11 mg/kg once weekly for three weeks (days 10, 17 and 22 post tumor cell inoculation) administered intravenously. The third group received single-agent DOXIL® at a dose of 11 mg/kg (single injection on day 10 post tumor cell inoculation) administered intravenously. A fourth group was treated with the combination of lorvotuzumab mertansine and DOXIL® with the same doses and routes of administration as for individual treatments. Tumor growth was monitored by measuring tumor size twice per week. Tumor size was calculated with the formula: length×width×height× 1/2.

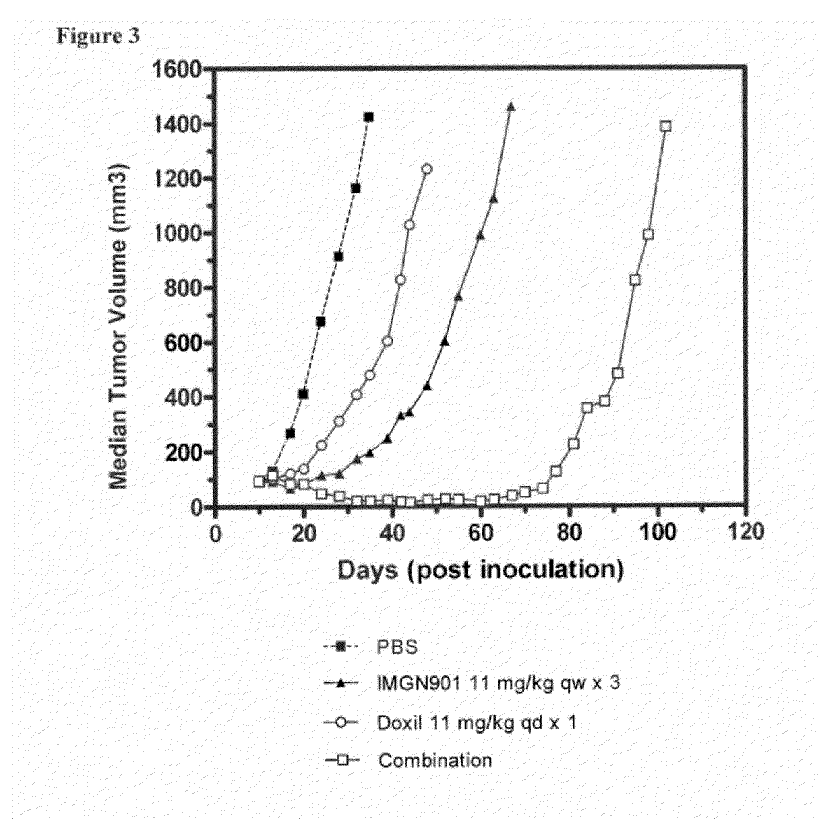

FIG. 3. Single-agent lorvotuzumab mertansine was active against COLO 720E xenografts in this study, with a T/C of 14%, with a CR in one of six mice, remaining tumor-free to the end of the study (day 118). Single-agent DOXIL® treatment was also active (T/C=35%) with no tumor regressions. The combination of lorvotuzumab mertansine with DOXIL® was highly active by NCl standards (T/C=3%) with PRs in all mice (six of six) and CRs in two of six mice with one remaining tumor free until the end of the study.

### Provisos

It is to be appreciated that the Detailed Description section, and not the Abstract, is intended to be used to interpret the claims. The Abstract may set forth one or more but not all exemplary embodiments of the present invention as contemplated by the inventors, and thus, is not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted as by a person of ordinary skill in the most closely related art in light of the teachings and guidance.

The breadth and scope of the present invention should not be limited by any of the above-described exemplary embodiments.

All publications, patents, and patent applications mentioned in this specification are herein incorporated by reference to the same extent as if each independent publication, patent, or patent application was specifically and individually indicated to be incorporated by reference.

1) A method of treating ovarian, peritoneal, or fallopian tube cancer comprising administering to a subject in need thereof a therapeutically useful amount of:
(i) an antibody or fragment thereof which specifically binds CD56, and
(ii) a chemotherapeutic agent,
wherein said antibody or fragment thereof is linked to a cytotoxic compound, and
wherein said chemotherapeutic agent is selected from the group consisting of:
(a) a gemcitabine compound;
(b) a topotecan compound; and
(c) a doxorubicin compound,
wherein said method provides a synergistic therapeutic effect in the treatment of ovarian, peritoneal, or fallopian tube cancer.

2) (canceled)

3) A pharmaceutical kit comprising:
(i) an antibody or fragment thereof which specifically binds CD56, and
(ii) a chemotherapeutic agent,
wherein said antibody or fragment thereof is linked to a cytotoxic compound, and
wherein said chemotherapeutic agent is selected from the group consisting of:
(a) a gemcitabine compound;
(b) a topotecan compound; and
(c) a doxorubicin compound.

4) A method of developing or formulating an ovarian, peritoneal, or fallopian tube cancer treatment regimen comprising administering:
(i) an antibody or fragment thereof which specifically binds CD56, and
(ii) a chemotherapeutic agent
to a non-human mammal, wherein said antibody or fragment thereof is linked to a cytotoxic compound, and wherein said chemotherapeutic agent is selected from the group consisting of:
(a) a gemcitabine compound;
(b) a topotecan compound; and
(c) a doxorubicin compound,
wherein said method comprises administration of said antibody or fragment thereof and said chemotherapeutic agent at varying doses and/or intervals, wherein the therapeutic effect of two or more varying doses and/or intervals are compared to identify a preferred treatment regimen.

5) The method of claim 4, further comprising administering said antibody or fragment thereof and said chemotherapeutic agent to a subject in need thereof in the preferred regimen.

6) (canceled)

7) The method of claim 1, wherein said method comprises administering said antibody and said chemotherapeutic agent to a human in need thereof.

8) (canceled)

9) (canceled)

10) (canceled)

11) (canceled)

12) The method of claim 1, wherein said antibody or fragment thereof or said chemotherapeutic agent is administered at a dose that would be non-therapeutic if administered alone.

13) (canceled)

14) The method of claim 1, wherein said cytotoxic compound is an anti-mitotic agent.

15) The method of claim 14, wherein said anti-mitotic agent is a maytansinoid.

16) The method of claim 15, wherein said maytansinoid is DM1.

17) The method of claim 1, wherein said antibody or fragment thereof is linked to said cytotoxic compound by use of a linker molecule selected from the group consisting of:
a) N-succinimidyl 4-(2-pyridyldithio)pentanoate (SPP);
b) N-succinimidyl 3-(2-pyridyldithio)propionate (SPDP); and
c) N-succinimidyl 4-(2-pyridyldithio)butanoate (SPDB).

18) The method of claim 17, wherein said cytotoxic compound is DM1, and said linker molecule is SPP.

19) The method of claim 1, wherein said chemotherapeutic agent is a gemcitabine compound.

20) (canceled)

21) The method of claim 1, wherein said chemotherapeutic agent is a topotecan compound.

22) (canceled)

23) The method of claim 1, wherein said chemotherapeutic agent is a doxorubicin compound.

24) (canceled)

25) The method of claim 1, wherein said antibody or fragment thereof is a humanized antibody or a fragment thereof.

26) The method of claim 25, wherein said antibody or fragment thereof is huN901.

27) The method of claim 26, wherein said antibody or fragment thereof linked to a cytotoxic compound is lorvotuzumab mertansine.

28) (canceled)

29) The method of claim 1, wherein said ovarian cancer is advanced, refractory, or recurrent ovarian cancer.

30) (canceled)

31) (canceled)

32) The method of claim 1, wherein said cancer is platinum-resistant.

33) The method of claim 1, wherein said cancer is platinum-sensitive.

34) (canceled)


SEQUENCE LISTING (2 sequences)

SEQ ID NO: 1 - protein, 219 aa, artificial sequence
  gN901LCv1.1 light chain
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, artificial sequence
  gN901HCv1.1 heavy chain
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK